(19) United States
(12) Patent Application Publication
(10) Pub. No.: US 2007/0255134 A1
(43) Pub. Date: Nov. 1, 2007

(54) Method And Device For Detecting A Dye Bolus Injected Into The Body Of A Living Being

(76) Inventors: Adam Liebert, Piaseczno (PL); Heidrun Wabnitz, Mahlow (DE); Rainer MacDonald, Berlin (DE); Jens Steinbrink, Berlin (DE); Hellmuth Obrig, Berlin (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 10/599,292

(22) PCT Filed: Mar. 22, 2005
(86) PCT No.: PCT/DE05/00539

§ 371 Date: Sep. 25, 2006

(30) Foreign Application Priority Data
Mar. 26, 2004 (DE) 10 2004 015 682.4

(51) Int. Cl. A61B 6/00

(52) U.S. Cl. 600/431

(57) ABSTRACT

Determinations of perfusion on the body of a living being are possible by detecting a dye bolus injected into the body by irradiating radiation into the body and detecting the response radiation occurring on the surface of the body. The aim of the invention is to make it possible to reliably carry out these determinations with a simple compact and transportable device. To this end, a fluorescent dye is injected, and optical excitation radiation is irradiated into the body, and a temporal relation between a fluorescent radiation, which is triggered by the excitation radiation, and the excitation radiation is measured.

The invention relates to a method for detecting a dye bolus injected into the body of a living being, by irradiating optical radiation into the body and detecting a response radiation occurring on the surface of the body.

The invention also relates to a device for detecting a dye bolus injected into the body of a living being, with an optical radiation source for irradiating an optical radiation into the body, and with a detection arrangement for detecting a response radiation emanating from the body.

It is known to examine the blood perfusion of tissues by means of a contrast agent bolus. The contrast agent is injected within a short time period, and the time characteristics of the contrast agent through the body are monitored. In cases of reduced blood perfusion, for example as a result of partial occlusion of arteries, the bolus takes longer to reach a target area.

The standard technique for non-invasive assessment of blood perfusion with the aid of a contrast agent bolus is magnetic resonance imaging using Gd-DTPA (gadolinium diethylenetriamine pentaacetic acid).

Another known method is positron emission tomography (PET) using radioisotopes.

Because of the measurement devices needed, these known methods require considerable outlay in terms of equipment and are expensive, and they cannot therefore be used for continuous monitoring of patients at the bedside, in the operating theater or on the intensive care ward of hospitals.

Studies have already been carried out into permitting non-invasive assessment of blood perfusion by means of optical contrast agents. An example of a dye approved for use on humans is indocyanine green (ICG). A dye such as this can be detected in tissue with the aid of diffuse near-infrared reflectometry or diffuse near-infrared spectroscopy, so that the time characteristics of a dye bolus can be monitored in a similar way to that in the abovementioned methods. Optical measurement methods would have the advantage of being able to be carried out with less outlay and with compact and transportable measurement devices. A particular need exists for determination of vascular occlusions in the brain, so that studies have been conducted into whether the optical method can be carried out on the head. The technique of near-infrared spectroscopy of the head uses continuous light that is guided by an optical fiber or fiber bundle to the surface of the head. The diffuse reflection of the near-infrared light is measured at a distance of a few centimeters (e.g. 3 cm) on the surface of the head. The detected light passes through various layers, particularly skin and bone, and in doing so is scattered and absorbed. In adults, the tissue layers lying across the cerebral cortex have a considerable thickness (approximately 1 cm), with the result that only a small proportion of the irradiated light reaches the underlying cortex, whose perfusion is the main point of interest. Using this approach, it is therefore not possible to obtain a measurement variable that contains information exclusively on the cortex.

The dye ICG that can be used, for example, is a blood pool agent, i.e. the dye remains in the blood and does not bind to tissue. Its concentration in the body decreases again according to the rate by which it is broken down by the liver. The dye is injected intravenously and passes through the right ventricle of the heart into the pulmonary circulation, and then through the left ventricle of the heart into the systemic circulation, and consequently into the cortex and also into the (extracerebral) layers of skin and bone lying over it. On entering the head, the dye bolus has a time width of 10 seconds. It enters the cortex earlier than it enters the extracerebral layers. With an intact blood-brain barrier, it rapidly leaves the cortex again, whereas the washout in the skin, for example, takes place much more slowly. These kinetics are also known from nuclear magnetic resonance tomography with contrast agent (Gd-DTPA). The arrival of the bolus at a specific area of skin is dependent on the local vessel distribution and is therefore not homogeneous. If the measurement signal contains considerable signal components from the skin, the kinetics of the contrast agent bolus cannot therefore supply any relevant information concerning the blood perfusion of the cortex.

Methods have been developed and made known that are designed to detect absorption changes with depth resolution and, by this means, to permit a separation of signal components from the cortex and from the layers lying over the cortex. For this purpose, short laser pulses have also been used for detecting the diffuse reflection with time resolution. In this case, the interval of the response signal in its time distribution has been taken into account, for example by having determined the integral, a mean interval or the time variance (width of the response curve). An exact separation of signal components originating from intracerebral and extracerebral layers is also not possible in these techniques. This is because the diffuse reflection is affected by all the changes in the absorption and scatter properties of the tissue penetrated by radiation, in other words not just by the absorption changes caused by the dye bolus. This concerns in particular physiological influences, for example heart beat and respiration, which thus make it difficult to analyze the signal response to the bolus. In addition, the diffuse reflection through the dye bolus changes to the order of 10%. The uncertainties caused by the abovementioned physiological influences always relate, however, to the full size of the signal, so that the dynamic range of the useful signal is considerably compromised.

Consideration has been given to carrying out further analysis for determination of absorption changes with depth resolution. This requires a knowledge of the absorption coefficients and scattering coefficients of the different types of tissues. In practice, however, at least some of these cannot be determined for an examination carried out on a living being.

There is therefore a considerable need for allowing detection of an injected dye bolus using a simple, compact and transportable device.

According to the invention, this object is achieved by a method of the type mentioned at the outset, characterized in that a fluorescent dye is injected, an optical excitation radiation is irradiated into the body, and a temporal relation between a fluorescent radiation, which is triggered by the excitation radiation, and the excitation radiation is measured.

Said object is also achieved by means of a device of the type mentioned at the outset, characterized in that the optical radiation source is designed to emit pulses of an excitation radiation with a first frequency, and the detection arrangement is designed to detect a response radiation with a second frequency different than the first frequency and to determine a temporal relation between the emitted excitation radiation and at least part of the detected response radiation.

According to the invention, therefore, a fluorescent radiation is detected which is generated by a preferably pulsed excitation radiation in the dye bolus, on account of its fluorescent property. A response signal with time resolution is measured, at least the interval of part of the response signal from the triggering excitation pulse being determined as a measure of the flight time of the fluorescent signal through the tissue layers. The pulsed excitation radiation preferably has a pulse duration of a few picoseconds (ps). The time resolution of the generated fluorescence signal lies in the nanosecond range or preferably in the picosecond range.

The detection of the fluorescent radiation has the advantage that it is specific to the injected dye, in other words is only present when the injected dye is located in the tissue penetrated by radiation. In principle, therefore, other signal profiles arise for the fluorescent radiation than in the diffuse reflection. In addition, as regards the intervals of the fluorescent light from the generating excitation pulse (according to the flight time of the fluorescence photons through the tissue), there are peculiarities that make it possible to differentiate between intracerebral and extracerebral bolus responses. Thus, for example, the mean flight time of the fluorescent light increases at the start of the dye bolus, after which it falls off sharply. Such a profile is not shown by reflected light. In addition, the fluorescence intensity can also be monitored over a much greater dynamic range than can the diffuse reflection, because the fluorescence intensity is not superposed by a necessarily existing background signal. According to the invention, a dye is used that is nonspecific, in other words does not bind to specific cells, as is the case, for example, with fluorescence markers that bind to certain cancer cells. The dye used is preferably a blood pool agent.

The use of fluorescent dyes for tissue examination is already known in principle. The present invention differs from this in terms of the time-resolved determination of the fluorescence response to an excitation pulse, with the peculiarities arising from the detection of the dye bolus.

The invention can be used not just for examination in the area of the brain (although this is of great relevance), but also for assessing perfusion in other organs lying beneath the surface of the body, in particular also the lungs.

The invention permits numerous other determinations, for example of the thickness of the extracerebral tissue layer and the permeability of the blood-brain barrier, based on an analysis of the kinetics of the washout of the dye.

If necessary, the invention can be refined using several emitter and receiver optodes, in which case the several optodes can also be arranged at different distances.

The measurement of the temporal relation or of the time profile of the fluorescence response can also be carried out using high-frequency modulated light, if the modulation depth and the phase in the response signal are determined.

The fluorescence measurement can be refined by spectral analysis of the fluorescence signal. Special dyes change their fluorescence frequency when accumulated in the blood. The resulting change in frequency can be used to reach conclusions regarding the origin of the fluorescent radiation from dye accumulated in the blood.

It is particularly expedient if the measurement, according to the invention, of the fluorescence response is combined with a measurement, known per se, of the diffuse reflection of the excitation radiation. The information obtainable therefrom, using known evaluation methods, can be used to supplement and verify the information determined from the measurement, according to the invention, of the fluorescence response.

The invention is explained in more detail below on the basis of illustrative embodiment depicted in the drawing, in which:

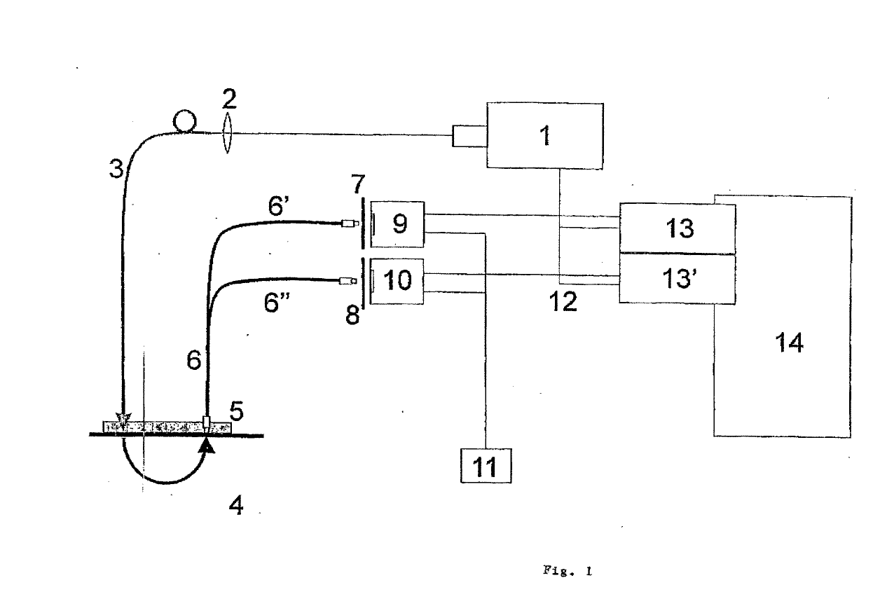

FIG. 1 shows a schematic representation of an illustrative embodiment of a device according to the invention,

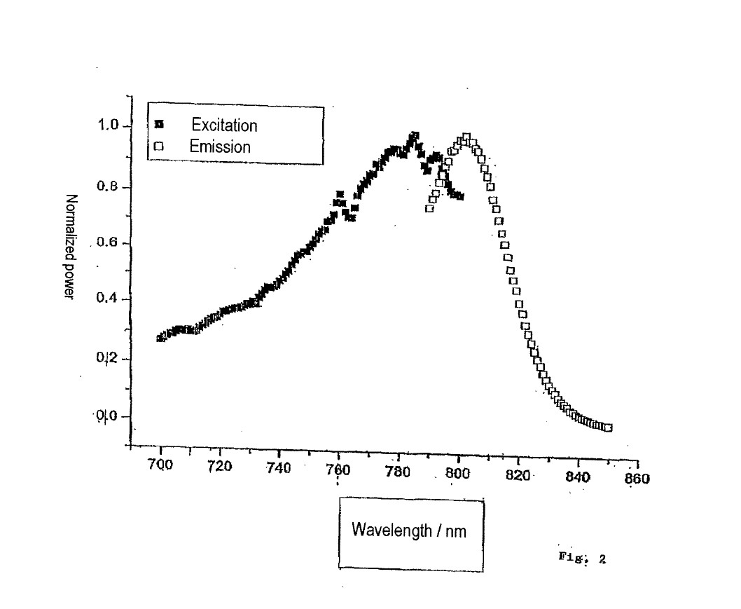

FIG. 2 shows a graph illustrating the spectrum of the excitation wavelengths and emission wavelengths for the dye ICG,

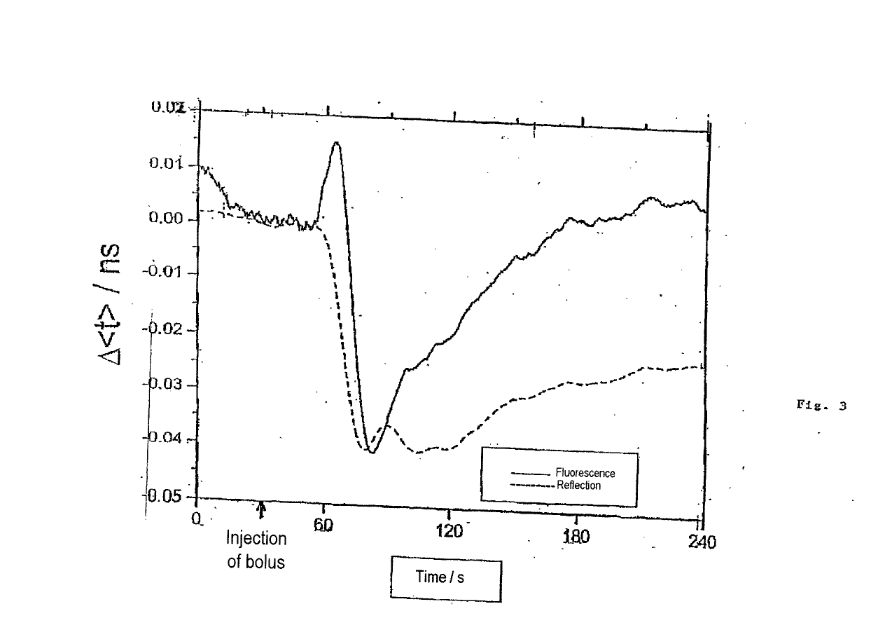

FIG. 3 shows a representation of the mean photon flight time of the fluorescence photons and of the reflected photons during transit of the dye bolus,

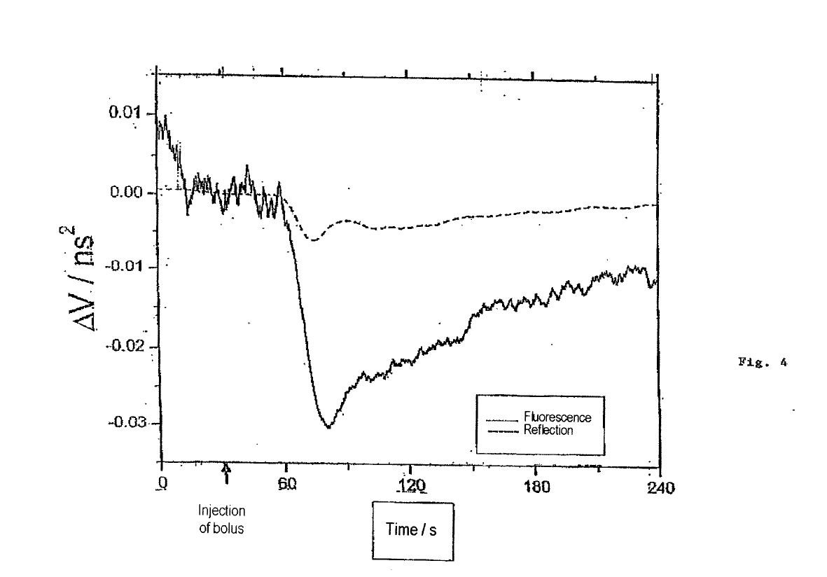

FIG. 4 shows a representation of the change in variance of the detected flight time for the fluorescence photons and the reflected photons.

FIG. 1 shows a semiconductor laser 1 which emits light pulses with a width in the picosecond range and a wavelength of 780 nm. The output beam is coupled via a lens 2 into a fiber-optic 3 and directed to a body 4 of a living being to be examined. The fiber-optic 3 ends in a holder 5, which also receives a detection fiber-optic bundle 6. The fiber-optics 3, 6 can be brought into contact, through the holder 5, with the skin of the body 4 that is to be examined, and they are expediently perpendicular to the surface of the skin.

The fiber-optic bundle 6 divides into a first detection fiber-optic 6′ and a second detection fiber-optic 6″.

The first detection fiber-optic 6′ is provided with a high-pass filter 7 with which the wavelength of the semiconductor laser 1 can be suppressed.

The second detection fiber-optic 6″ has an attenuation filter 8. Detectors 9, 10 in the form of photo-multipliers are attached to both detection fiber-optics 6′, 6″ respectively, both of these detectors 9, 10 being supplied with the required high voltage by a high-voltage source 11. The photomultipliers can detect individual photon pulses. Their outputs are connected to an electronic counter 13, which is started up by a pulse transmitted from the semiconductor laser 1 via starter inputs 12, in order to determine the interval of the photons, detected in the detectors 9, 10, from the excitation pulse of the semiconductor laser 1. The photon flight times thus determined reach a computer 14, which can be in the form of a personal computer PC.

The device shown in FIG. 1 is used to detect an injected dye bolus. The dye bolus is injected for example into the brachial vein. An example of a suitable fluorescent dye is indocyanine green (ICG).

FIG. 2 shows the excitation spectrum for ICG, its maximum lying at about 780 nm. FIG. 2 also shows the emission spectrum of ICG, its maximum lying at about 810 nm.

The excitation wavelength of 780 nm used here thus lies in the excitation maximum of ICG. The measurements of the fluorescent radiation were carried out using a filter 7 whose transmit value starts at about 820 nm, in order to ensure a safe distance from the excitation radiation.

The structure in FIG. 1 illustrates that, in addition to the fluorescence measurement in the detector 9, a reflection measurement in the detector 10 is also carried out. The photon flight times are measured in both cases, that is to say the interval between the emitted excitation pulse of the semiconductor laser 1 and the response photons detected in the detectors 9, 10.

FIG. 3 shows the measured mean flight time for the fluorescence photons and for the photons of the reflected light during transit of the dye bolus, which passes through the cerebral cortex about 60 seconds after the injection.

It will be seen from FIG. 3 that, at the start of the detection of the dye bolus, the flight time of the fluorescence photons rises significantly, and that it drops abruptly after the end of the dye bolus, which has a width of about 10 seconds, thereafter rising again when the dye enters the extracerebral layers.

By contrast, the measurement of the reflected light during transit of the dye bolus shows only a decrease in the flight time, which thereafter slowly rises again. The curves show that measurement only of the reflected photons does not permit a clear localization of the width of the bolus, since effects of the extracerebral tissue are immediately superposed.

FIG. 4 also shows that the variance, that is to say the deviations in the measurements of the flight time during transit of the bolus, decreases significantly for the fluorescence photons, whereas practically no such effect can be observed for the reflected light.

It will be immediately apparent from these examples that the fluorescence photons behave very differently than the reflected light during transit of the dye bolus, and better differentiation, for example between intracerebral and extracerebral effects, is therefore permitted.

1. A method for detecting a dye bolus injected into the body of a living being, by irradiating optical radiation into the body (4) and detecting a response radiation occurring on the surface of the body, characterized in that a fluorescent dye is injected, an optical excitation radiation is irradiated into the body, and a temporal relation between a fluorescent radiation, which is triggered by the excitation radiation, and the excitation radiation is measured.

2. The method as claimed in claim 1, characterized in that the excitation radiation is emitted as a short pulse.

3. The method as claimed in claim 1, characterized in that a time profile of the fluorescent radiation triggered by the excitation radiation is determined.

4. The method as claimed in claim 1, characterized in that, for detection of the fluorescent radiation, the frequency of the excitation radiation is blocked off by filtering.

5. The method as claimed in claim 1, characterized in that a detection of the reflected excitation radiation is carried out simultaneously and in parallel.

6. The method as claimed in claim 5, characterized in that the detection of the reflected excitation radiation is likewise carried out with time resolution.

7. The method as claimed in claim 1, characterized in that the detected fluorescent radiation is evaluated by assessing the distribution of the measured temporal relation.

8. The method as claimed in claim 7, characterized in that a rise in the distribution is used as an indicator for the start of the dye bolus.

9. The method as claimed in claim 1, characterized in that the excitation radiation is irradiated into the body (4) at the head in order to examine the brain.

10. The method as claimed in claim 1, characterized in that the excitation radiation is irradiated into the body (4) in the area of the lungs.

11. A device for detecting a dye bolus injected into the body (4) of a living being, with an optical radiation source (1) for irradiating an optical radiation into the body (4), and with a detection arrangement (6-16) for detecting a response radiation emanating from the body (4), characterized in that the optical radiation source (1) is designed to emit an excitation radiation with a first frequency, and the detection arrangement is designed to detect a response radiation with a second frequency different than the first frequency and to determine a temporal relation between the emitted excitation radiation and at least part of the detected response radiation.

12. The device as claimed in claim 11, characterized in that the optical radiation source (1) operates in pulsed mode.

13. The device as claimed in claim 11, characterized in that the detection arrangement (6-14) is designed to detect a time profile of the fluorescent radiation triggered by a pulse of the excitation radiation.

14. The device as claimed in claim 11, characterized in that the detection arrangement (6-14) has an optical filter (7) for blocking off the excitation radiation.

15. The device as claimed in claim 11, characterized in that the detection arrangement (6-14) has an additional detector branch (6″, 8, 10) for detection of reflected excitation radiation.

16. The device as claimed in claim 11, characterized in that the detection arrangement (6-14) has an evaluation unit (14) for temporal changes of the measured temporal relation.
